(19) United States
(12) Patent Application Publication
(10) Pub. No.: US 2009/0208941 A1
(43) Pub. Date: Aug. 20, 2009

(54) Method for investigating cytosine methylations in dna

(75) Inventors: Kurt Berlin, Stahnsdorf (DE); Matthias Schuster, Berlin (DE); Philipp Schatz, Berlin (DE)

Correspondence Address: KRIEGSMAN & KRIEGSMAN, 30 TURNPIKE ROAD, SUITE 9, SOUTHBOROUGH, MA, 01772, US

(73) Assignee: EPIGENOMICS AG, BERLIN (DE)

(21) Appl. No.: 11/989,081

(22) PCT Filed: Jul. 19, 2006
(86) PCT No.: PCT/EP2006/064408

§ 371 Date: Mar. 30, 2009

(30) Foreign Application Priority Data
Jul. 19, 2005 (DE) 10 2005 034 628.6

(51) Int. Cl. C12Q 1/68

(52) U.S. Cl. 435/6

(57) ABSTRACT

A method for sensitive and specific detection of cytosine methylation is described wherein the DNA to be analyzed is subjected to initial enrichment. More specifically, said enrichment can be effected in a methylation-specific, sequence-specific or origin-specific fashion. Thereafter, the enriched DNA is converted in a methylation-specific fashion. The converted DNA can be analyzed using various methods, particularly real-time PCR methods.

The present invention relates to a method for the detection of 5-methylcytosine in DNA. 5-Methylcytosine is the most common covalently modified base in the DNA of eukaryotic cells. It plays an important biological role e.g. in the regulation of transcription, in genetic imprinting and in tumorigenesis (for a review see: Millar et al.: Five not four: History and significance of the fifth base. In: The Epigenome, S. Beck and A. Olek (eds.), Wiley-VCH Verlag Weinheim 2003, pp. 3-20). The identification of 5-methylcytosine as an element of genetic information is therefore of considerable interest. However, the detection of methylation is difficult because cytosine and 5-methylcytosine have the same base pairing behavior. For this reason, many of the conventional detection methods based on hybridization cannot distinguish between cytosine and methylcytosine. In addition, the methylation information is completely lost during PCR amplification.

Essentially, conventional methods for methylation analysis operate according to two different principles. On the one hand, methylation-specific restriction enzymes are used, and, on the other hand, selective chemical conversion of non-methylated cytosines into uracil takes place (so-called bisulfite treatment, see e.g. DE 101 54 317 A1; DE 100 29 915 A1).

The treatment with methylation-specific restriction enzymes is restricted to particular sequences as a result of the sequence specificity of these enzymes, so that a bisulfite treatment is performed in most uses (for a review see DE 100 29 915 A1, p. 2, lines 35-46). Frequently, the chemically pretreated DNA is then amplified and can be analyzed in various ways (for a review see WO 02/072880 pp. 1ff). Methods which can detect methylation in a sensitive and quantitative manner are of great interest. This is true because of the important role of cytosine methylation in the development of cancer, particularly with respect to diagnostic uses. Of particular importance are methods allowing detection of aberrant methylation patterns in body fluids, such as serum, because unlike unstable RNA, DNA is frequently found in body fluids. The DNA concentration in blood is even higher in destructive pathological processes such as cancerous diseases. Thus, cancer diagnosis via methylation analysis of tumor DNA present in body fluids is possible and has been described repeatedly (see e.g. Palmisano et al.: Predicting lung cancer by detecting aberrant promoter methylation in sputum. Cancer Res. 2000 Nov 1; 60 (21): 5954-8). However, one particular problem is that in addition to the DNA having the methylation pattern typical of a disease, a large quantity of DNA of identical sequence but different methylation pattern is also present in body fluids. The diagnostic methods must therefore be able to detect small quantities of specifically methylated DNA in the presence of a strong background of DNA of identical sequence but different methylation pattern (hereinafter referred to as background DNA).

Conventional methods of analyzing methylation solve this problem only to a limited extent. The chemically pretreated DNA is normally amplified using a PCR method. In this event, selective amplification of methylated DNA only (or, in the reverse approach, non-methylated DNA only) is to be ensured by using methylation-specific primers or blockers. The use of methylation-specific primers is known as so-called “methylation-sensitive PCR” (“MSP”; Herman et al.: Methylation-specific PCR: a novel PCR assay for methylation status of CpG islands. Proc Natl Acad Sci USA, 1996 Sep 3; 93 (18): 9821-6). A comparably sensitive method is the so-called “Heavy Methyl” method wherein specific amplification of originally methylated (or non-methylated) DNA only is achieved by using methylation-specific blocker oligomers (for a review see WO 02/072880). Both MSP and Heavy Methyl can be used as quantifiable real-time variants. They allow detection of the methylation status of a few positions directly during the course of a PCR without requiring subsequent analysis of the products (“MethyLight”: WO 00/70090; U.S. Pat. No. 6,331,393). One such embodiment is the “Taqman” method which uses probe molecules bearing a fluorescent dye-quencher pair. The probes hybridize to the amplified products in a sequence-specific manner, undergoing degradation during the next amplification cycle as a result of the exonuclease activity of the polymerase. By separating quencher and dye, a detectable fluorescence signal is produced (see e.g. Eads et al.: MethyLight: a high-throughput assay to measure DNA methylation. Nucleic Acids Res. 2000 Apr 15; 28(8): E32). Another MethyLight embodiment is the so-called Lightcycler method which uses two different probes hybridizing to the amplified product in close vicinity to each other to generate a detectable signal via fluorescence resonance energy transfer (FRET).

However, the applicability of these methods for sensitive and specific detection of methylated DNA in the presence of a vast background of non-methylated DNA is limited. Thus, there is a risk of false-positive results arising via non-specific amplification of background DNA. However, false-positive signals represent one of the most significant problems when using the methylation technology in cancer early recognition. Increasing the specificity of methylation detection therefore represents an important step in the development of adequate early recognition tests. In this way, reliable commercial utilization of methylation analysis in the sector of tumor early diagnostics is facilitated.

To increase the specificity of methylation detection, well-known methods use primer or blocker sequences for amplification which include a plurality of methylation-specific positions. However, such sequence requirements only allow detection of sequences having a large number of CpG positions within a narrow sequence region. Said sequence requirements limit the applicability of such methods.

In view of the above-mentioned biological and medical relevance of cytosine methylation and the prior art drawbacks set forth above, there is a great technical need for the development of efficient methods for sensitive and specific methylation analysis. A surprisingly simple method enabling higher sensitivity and specificity in detecting methylation will be described below.

According to the invention, the DNA to be amplified is enriched prior to amplification. In a preferred fashion, enrichment is effected prior to bisulfite conversion, but can also be carried out thereafter. As a result of enrichment, the disease-specific DNA is easier to detect. According to the invention, the enrichment is preferably effected according to three different principles. Firstly, the enrichment can be performed in a methylation-specific fashion, that is to say, DNA of a particular methylation status is concentrated. For example, this can be done using methylcytosine-binding proteins (see e.g. Cross et al., Nat Genet. 1994 Mar; 6(3): 236-44). In a second embodiment of the invention, sequence-specific pre-purification is effected wherein only particular sequences are purified, e.g. those where a modified methylation status is known to associated with a disease. In a third embodiment of the invention, enrichment is effected in an origin-specific manner. Thus, for example, the DNA can be isolated specifically from tumor cells and subsequently subjected to further isolation. Preferred embodiments of the invention combine the above three principles. In general, purification is followed by bisulfite conversion and amplification/detection. If enrichment is performed subsequent to bisulfite conversion, sequence-specific and methylation-specific purifications are effected according to the same principle because the methylation information is converted into sequence information as a result of bisulfite treatment. Enrichment of the DNA to be detected reduces the risk of false-positive results, thereby allowing more specific detection of methylated cytosine positions.

Some of the enrichment methods of the invention have already been described in the context with methylation analysis. Thus, for example, purification of DNA with a methylcytosine-binding antibody (e.g. through an antibody column: Fisher et al., Nucleic Acids Res. 2004 Jan 9; 32(1): 287-97) prior to methylation analysis is well-known.

Nevertheless, the above enrichment method has only been used so far to identify hitherto unknown methylation positions. In contrast, the use of this method in a sensitive detection of cytosine positions already known to be associated with a disease still remains unknown.

As used hereinafter, sensitive detection is understood to be analysis allowing detection of 100 or less copies of methylated DNA in the presence of a background of thousands of copies of unmethylated or low-methylated DNA.

In view of the exceptional relevance of methylation analysis to tumor early diagnostics and the disadvantages of well-known methods, the establishment of this advantageous, new, and surprisingly simple technology represents an important technical progress.

The method according to the invention is a method for sensitive detection of cytosine methylation. As understood hereinafter, this concerns methods capable of detecting specific methylation positions in DNA with high sensitivity. As used hereinafter, sensitive detection is understood to be analysis allowing detection of 100 or less copies of methylated DNA in the presence of a background of thousands of copies of unmethylated or low-methylated DNA.

In the methods for sensitive detection, the cytosine positions to be analyzed are already known. Also, the type of biological condition, e.g. type of disease, correlating with the methylation status of these positions is already known. The situation is different in so-called “discovery methods”. As understood hereinafter, these are methods used for identification or more detailed characterization of methylation positions suspected to assume a particular biological role. The term “method for sensitive detection of cytosine methylation” is not intended to comprise these discovery methods.

The inventive method for sensitive methylation analysis proceeds in the following steps:
1) collecting a biological sample,
2) enrichment of the DNA to be detected,
3) methylation-specific conversion of the DNA by chemical or enzymatic treatment,
4) amplification of the converted DNA,
5) analyzing the amplified products.

In an alternative embodiment, enrichment is effected only after conversion of the DNA. The method therefore proceeds in the following steps:
1) collecting a biological sample,
2) methylation-specific conversion of the DNA by chemical or enzymatic treatment,
3) enrichment of the DNA to be detected,
4) amplification of the enriched DN,
5) analyzing the amplified products.

In the first step of the method according to the invention, a biological sample is collected from which the DNA is obtained. The biological sample can be derived from various sources, depending on the diagnostic or scientific problem. For diagnostic problems, tissue samples, but also body fluids, especially serum, are preferably used as starting materials. It is also possible to use sputum, stool, urine or cerebrospinal fluid. Initially, the DNA is preferably isolated according to standard procedures, e.g. from blood, using the Qiagen UltraSens DNA extraction kit.

In the second step of the method according to the invention, the DNA to be detected is enriched. “DNA to be detected” is understood to be that sequence whose methylation status is to be analyzed. Enrichment is understood to be a process wherein the concentration of the DNA to be detected is increased compared to other DNA. Depending on the enrichment process, said other DNA is background DNA (DNA of identical sequence, but different methylation status compared to the DNA to be detected), DNA having a different sequence, or DNA of different origin.

In a preferred embodiment of the method according to the invention, enrichment is effected in a methylation-specific fashion, with DNA to be detected being enriched with respect to the background DNA. Methylation-specific enrichment can be effected in various ways. Essentially, the DNA is contacted with substances specifically binding methylated or unmethylated sequences, which binding can be both sequence-specific and sequence-unspecific (via methylated cytosines only). Following binding to the substances, bound DNA can be separated from unbound DNA. Depending on whether the DNA to be detected is methylated or unmethylated, the bound or unbound fraction can be subjected to further analysis.

As described above, methylation-specific binding of the DNA to particular substances and subsequent separation of bound and unbound DNA are essential to methylation-specific enrichment. The term “methylation-specific enrichment” is not intended to comprise any methods wherein the background DNA is degraded by means of methylation-specific restriction enzymes. Such a method has already been described in the patent application DE 10 2005 011 398.2 and is not intended to form the contents of the present application. While methylation-specific binding of DNA to the restriction enzyme takes place in this case as well, such binding merely is a transitory state on the way to enzymatic cutting. In contrast, the method according to the invention involves separation of bound (uncut) DNA from unbound DNA.

Various substances binding to the DNA in a methylation-specific fashion are possible in the method according to the invention.

In a preferred embodiment, enrichment is performed using proteins binding to the DNA in a methylation-specific fashion. Several proteins of this type are known, e.g. MeCP2, MBD1, MBD2, MBD4 and Kaiso (for a review see: Shiraishi et al., Methyl-CpG binding domain column chromatography as a tool for the analysis of genomic DNA methylation. Anal Biochem. 2004 Jun 1; 329(1): 1-10; incorporated by reference in its entirety; Henrich et Tweedie: The methyl-CpG binding domain and the evolving role of DNA methylation in animals. Trend Genet. 2003 May; 19(5): 269-77; incorporated by reference in its entirety.

By using proteins that bind to the DNA in a methylation-specific fashion, it is possible to perform the methylation-specific enrichment in various ways. Thus, it is possible to use proteins specifically binding methylated DNA, as well as proteins specifically binding unmethylated DNA. Furthermore, it is possible to bind the particular DNA which is to be detected afterwards. To this effect, unbound DNA is removed first, and bound DNA is subsequently detached from the protein. It is also possible to bind the background DNA to the proteins and subsequently remove it from the reaction batch.

Protein binding and separation of bound and unbound DNA can be effected in various ways. Thus, for example, it is possible to bind the proteins to a solid surface, e.g. in the form of a column (cf. Cross et al. 1994, Nature Genetics Vol. 6, 236-244). Unbound DNA can subsequently be removed by wash steps. Furthermore, it is possible to allow binding to the proteins to proceed in solution and subsequently separate the DNA-protein complexes from unbound DNA using common methods such as centrifugation or chromatography. A person skilled in the art is familiar with biochemical methods to be used, e.g. the use of biotinylated proteins or proteins provided with a histidine tag (e.g. Gretch et al. 1987, Anal Biochem Vol. 163, 270-7; Janknecht et al. 1991, Proc Natl Acad Sci USA Vol. 88, 8972-6).

In a particularly preferred embodiment, enrichment is performed using the so-called MBD column chromatography described in detail by Shiraishi et al. 2004, loc. cit., with explicit reference being made to this publication.

To this end, it is possible to use the methyl-CpG-binding domain of the MeCP2 protein, which binds specifically methylated, but no unmethylated or hemimethylated cytosines. The corresponding domain expressed in vitro can be bound to a modified agarose surface e.g. via additional histidine residues. The domain recognizes sequence-unspecifically methylated CpG positions. Binding of the methylated DNA to the column proceeds depending on the methylation level and density of the CpG positions. Thereafter, bound methylated DNA molecules can be eluted by increasing the salt concentration and subsequently analyzed (for details, see: Shiraishi et al. 2004, loc. cit.).

In addition, analyzing the unbound, unmethylated fraction is also possible.

Other proteins binding to DNA in a methylation-specific fashion, especially proteins binding to CpG positions in an sequence-specific manner, e.g. the Kaiso protein which recognizes symmetrically methylated CpGpCpG positions, can be used for enrichment according to the principle set forth above.

Apart from the above-mentioned MDB proteins, it is also possible, in principle, to use other proteins recognizing DNA in a methylation-specific fashion. These include e.g. restriction enzymes or methyl transferases. It is conceivable that those parts of said enzymes which are responsible for methylation-specific binding are used for enrichment without the corresponding active center.

In another preferred embodiment, enrichment proceeds via methylation-specific antibodies. Anti-5-methylcytosine antibodies are known and commercially available for quite some time (www.abcam.com; Abcam Inc; One Kendall Square; Bldg. 200, 3^{rd} Floor; Cambridge, Mass. 02139). These antibodies can also be bound on a column or can be bound to the DNA in solution according to well-known methods (see Fisher et al., loc. cit.).

For enrichment, it is also conceivable, though not preferred due to the experimental input, to perform a chromatin immunoprecipitation (ChIP) (details of this method are known to those skilled in the art and can be found e.g. in: Matarazzo et al.: In vivo analysis of DNA methylation patterns recognized by specific proteins: coupling CHIP and bisulfite analysis. Biotechniques. October; 37(4): 666-8, 670, 672-3, 2004).

Apart from proteins, other substances capable of methylation-specific binding to the DNA can also be used according to the invention. These include e.g. triplex-forming PNA or DNA oligomers. For example, appropriate oligomers have been described in detail in the patent application PCT/EP04/06534 (applicant: Epigenomics AG). Therein, descriptions including further references can be found, indicating in which way triplex-binding molecules can be utilized for the isolation of methylated DNA (so-called triple-helix affinity chromatography).

In a second particularly preferred embodiment, enrichment of the DNA to be analyzed is effected in a sequence-specific rather than a methylation-specific manner. In this way, specific enrichment of those sequences is possible whose methylation status is correlated with a disease or other biological problem, whereas other sequences are removed from the reaction batch. Sequence-specific enrichment is preferably effected via hybridization to complementary sequences. In a preferred fashion, the latter are bound to a surface, such as a solid phase, or to particles. Those skilled in the art are familiar with different variants of enrichment, e.g. using modified PNA or DNA oligomers that are e.g. immobilized, biotinylated or provided with magnetic particles (for example, Riccelli et al., Nucleic Acids Res. 2001 Feb 15; 29(4): 996-1004). Subsequently, the enriched sequences can be eluted and converted in a methylation-specific fashion. However, it is possible to perform the subsequent conversion, especially bisulfite conversion, directly on the surface (see below).

Finally, in a third particularly preferred embodiment, enrichment is effected according to the origin of the DNA. More specifically, this will be understood hereinafter to mean the cellular origin of the DNA. In a preferred embodiment, enrichment of the DNA is effected via isolation of a particular type of cells. In a particularly preferred fashion, cancer cells are isolated from body fluids, such as blood, urine or stool. In addition, it is also possible to enrich a particular type of cells, such as T cells, for future analysis of the DNA.

For isolation of cells, various methods are known to those skilled in the art, using e.g. fluorescence-activated or magnetically activated or immobilized antibodies, e.g. FACS or MACS (for a review see: FACS Sorting: Herzenberg et al., Clin Chem. 2002 October; 48(10): 1819-27; MACS as positive selection: Qin et al., World J Gastroenterol 2004 May 15; 10(10): 1480-1486; MACS as negative selection: Lara et al., Exp Hematol. 2004 October; 32(10): 891-904).

According to the invention, it is also preferred to isolate DNA bound to subcellular fragments, such as nucleosomes. Here, the basic idea is that more DNA still bound to cellular structures is released in destructive pathological processes. Such DNA can be enriched by using organelle-specific purification methods, e.g. a Nuclei Isolation Kit: Nuclei PURE Prep from Life Science Research (see also: Blobel et al. 1966 Science Vol. 154, 1662-5), or by enrichment of nucleosomes (Whitlock et al. 1976 Nucleic Acids Res Vol. 3, 2255-66). Specific purification of transcription complexes of single genes is also possible (Pindolia et al. 2005 J Mol Biol. Vol. 349, 922-32).

In preferred embodiments of the method according to the invention, the above-mentioned enrichment procedures are combined with each other. Thus, for example, it is possible to perform a methylation-specific enrichment first (using e.g. a methylation-specific antibody), followed by sequence-specific capturing (optionally after a fragmentation step).

In the third step of the method according to the invention, the enriched DNA is subjected to methylation-specific conversion, using a chemical or enzymatic treatment. In one aspect, methylation-specific restriction enzymes can be employed to this end, e.g. in such a way that the restriction site is located in a region to be amplified later. An amplified product can only be formed in those cases where the DNA has not been cut. Details of this principle are well-known to those skilled in the art (cf. e.g. DE 10 2004 02 97 002). In a preferred embodiment, however, the DNA is subjected to chemical or enzymatic conversion in such a way that unmethylated cytosine is converted into thymine or another base differing from cytosine in its base pairing behavior, while methylcytosine remains unchanged. In a preferred fashion, a chemical bisulfite treatment is performed. Various types of bisulfite conversion are known to those skilled in the art (see e.g. Frommer et al.: A genomic sequencing protocol that yields a positive display of 5-methylcytosine residues in individual DNA strands. Proc Natl Acad Sci USA 1992 Mar 1; 89(5): 1827-31; Olek, A modified and improved method for bisulphite based cytosine methylation analysis. Nucleic Acids Res. 1996 Dec 15; 24(24): 5064-6; DE 100 29 915; DE 100 29 915). In a particularly preferred fashion, bisulfite conversion is conducted in the presence of denaturing solvents, such as dioxane, and a free-radical scavenger (see DE 100 29 915). Other preferred embodiments of bisulfite conversion have been described in the German patent applications DE 103 47 396.3; DE 103 47 397.1; DE 103 47 400.5 and DE 103 47 399.8. Also, bisulfite conversion has been carried out on a solid phase (cf. PCT/DE02/04054; EP 03 019 321.3). If enrichment has been effected on a solid phase, it is therefore conceivable to perform the bisulfite conversion immediately thereafter, with no previous elution of DNA.

In another preferred embodiment the DNA is converted by enzymatic rather than chemical means. Conceivable in this respect is, for example, the use of cytidine deaminases which react unmethylated cytidines more rapidly than methylated cytidines. More recently, a corresponding enzyme has been identified (Bransteitter et al.: Activation-induced cytidine deaminase deaminates deoxycytidine on single-stranded DNA but requires the action of RNase. Proc Natl Acad Sci USA 2003 Apr 1; 100(7): 4102-7; cf.: German patent application 103 31 107.6).

In an alternative embodiment of the method according to the invention, enrichment is performed subsequent to DNA conversion. In this event, both sequence-specific and methylation-specific purification are effected according to the same principle because the methylation information has been converted into sequence information as a result of the bisulfite treatment. For sequence-specific purification, the methods described above can be employed, particularly using modified DNA or PNA oligomers. Explicit reference is made to the explanations above. In the fourth step of the method according to the invention, the converted DNA is amplified and subsequently analyzed. Thereafter, it is possible to conclude the methylation state of the DNA.

Various methods of amplifying DNA, such as ligase chain reactions, are known to those skilled in the art. In a preferred embodiment, however, the DNA is amplified via polymerase reaction. To this effect, various embodiments are conceivable, such as the use of isothermal amplification processes. However, polymerase chain reactions (PCR) are particularly preferred. In a most preferred embodiment, the PCR is performed using primers specifically binding only to previously methylated (or in the reverse approach: unmethylated) positions of the converted sequence. When using bisulfited DNA, this process is known as methylation-sensitive PCR (MSP). The process uses primers containing at least one 5′-CpG-3′ dinucleotide; those primers are preferred which bear at least three 5′-CpG-3′ positions, at least one of them being located at the 3′ end. Accordingly, 5′-TG-3′ or 5′-CA-3′ dinucleotides are required to amplify the unmethylated sequences or the counter-strands (cf.: Herman et al.: Methylation-specific PCR: a novel PCR assay for methylation status of CpG islands. Proc Natl Acad Sci USA 1996 Sep 3; 93(18): 9821-6).

For bisulfite-pretreated DNA, another particularly preferred embodiment is known under the name of “Heavy Methyl” method wherein specific amplification of originally methylated (or unmethylated) DNA only is achieved by using at least one methylation-specific blocker oligomer. The blocker binds to a 5′-CG-3′ (or 5′-TG-3′ dinucleotide or 5′-CA-3′) dinucleotide, thereby preventing amplification of background DNA. By selecting the polymerase or modifying the blocker oligomers, the above embodiment can be designed in such a way that degradation or extension of the blockers is minimized (for a review see: WO 02/072880; Cottrell et al., A real-time PCR assay for DNA methylation using methylation-specific blockers. Nucleic Acids Res. 2004 Jan 13; 32(1): e10).

The amplified products can be detected using conventional procedures, e.g. methods of length measurement such as gel electrophoresis, capillary gel electrophoresis and chromatography (e.g. HPLC). Mass spectrometry and methods of sequencing such as the Sanger method, Maxam-Gilbert method and sequencing by hybridization (SBH) can also be used. In a preferred embodiment the amplified products are detected using primer extension methods (see e.g.: Gonzalgo & Jones: Rapid quantitation of methylation differences at specific sites using methylation-sensitive single nucleotide primer extension (Ms-SNuPE). Nucleic Acids Res. 1997 Jun 15; 25(12): 2529-31; DE 100 10 282; DE 100 10 280).

In another preferred embodiment the amplified products are analyzed using hybridization to oligomer arrays (a review on array technology can be found in a special edition of: Nature Genetics Supplement, Volume 21, January 1999). On such an array, different oligomers can be arranged on a solid phase in the form of a right-angled or hexagonal grid. The solid phase surface is preferably composed out of silicon, glass, polystyrene, aluminum, steel, iron, copper, nickel, silver or gold. However, nitrocellulose and plastic materials, such as nylon, which can be present in the form of pellets or resin matrices, are also possible. The amplified products—e.g. fluorescence-labeled—are hybridized to the bound oligomers, and unbound fragments are removed. In an advantageous fashion, the oligomers hybridize via a section 12-22 bases in length to the DNA to be analyzed and comprise at least one CG, TG or CA dinucleotide. The fluorescence signals can be scanned and processed using software programs (see e.g. Adorjan et al., Tumour class prediction and discovery by microarray-based DNA methylation analysis. Nucleic Acids Res. 2002 Mar 1; 30(5): e21).

In a particularly preferred fashion the amplified products are analyzed using PCR real-time variants (cf.: Heid et al.: Real-time quantitative PCR. Genome Res. 1996 October; 6(10): 986-94, U.S. Pat. No. 6,331,393 “Methyl-Light”) wherein amplification is performed in the presence of a fluorescence-labeled reporter oligonucleotide that hybridizes to a 5′-CG-3′ dinucleotide (or 5′-TG-3′ or 5′-CA-3′ dinucleotide). The reporter oligonucleotide preferably binds to the DNA to be investigated, indicating amplification thereof by an increase or decrease in fluorescence. In a particularly advantageous fashion, the change in fluorescence is directly utilized for analysis and the methylation status is concluded from the fluorescence signal. A particularly preferred variant is the “Taqman™” method. In another particularly preferred embodiment, an additional fluorescence-labeled oligomer is used which hybridizes in close proximity to the first reporter oligonucleotide, which hybridization is detectable by means of fluorescence resonance energy transfer (“Lightcycler™” method). Those skilled in the art are familiar with further real-time variants using e.g. Molecular Beacons™ or Scorpion™ primers (cf.: DE 103 38 308). The corresponding variants also form part of this invention.

In another particularly preferred embodiment of the method according to the invention, a so-called QM assay is performed. The QM assay uses two methylation-unspecific primers and two different real-time probes, one of which is specific for the methylated state and the other one is specific for the unmethylated state. The QM assay allows excellent quantification of methylation states. The QM assay has been described in detail in the European patent application 04 090 213.2, to which explicit reference is made.

Another preferred embodiment of the invention is simultaneous amplification of several fragments using a multiplex PCR. In the design thereof care must be taken to avoid mutual complementarity of both the primers and the other oligonucleotides employed, so that high-level multiplexing in this case is more difficult than usual. However, owing to the different G and C content of the two DNA strands, enzymatically pretreated DNA is advantageous in that a forward primer can never act as a reverse primer, which in turn facilitates multiplexing and essentially compensates for the above-described disadvantage. Again, detection of the amplified products is possible via various methods, to which end e.g. the use of real-time variants is conceivable. For amplification of more than four genes, however, detection of the amplified products in some other way is recommended. To this effect, analysis via arrays is preferred (see above).

In a preferred embodiment of the method according to the invention, sensitive detection is effected without previous amplification. For example, this is possible when combining different purification methods. Also, chemical conversion of the DNA may not be required in this case. Thus, sequence-specific capturing can be performed first, e.g. by using a chip or by means of a specific transcription factor where the sequence to be investigated binds to, followed by detection of methylated sequences using (e.g. fluorescence—or radioactively) labeled methylation-specific antibodies. As a rule, it will be necessary to amplify the signal. This can be done by means of branched probes which, in the form of a tree structure, have both target-specific sequence sections and sequence portions for signal generation to which enzymatically labeled detection probes can bind (e.g. alkaline phosphatase). Alternatively, a plurality of signal cascades can be coupled (e.g. alkaline phosphatase with alcohol dehydrogenase and diaphorase, see: Johannsson 1985 Clin Chim Acta Vol. 148 119-24).

Alternatively, a methylation-specific antibody can be coupled with an oligonucleotide. The antibody binds to methylated sequences. A second oligonucleotide binds in a sequence-specific manner and, using a ligation reaction, can be bound to the oligonucleotide which is linked to the antibody. The new product being formed can be detected using a PCR or LCR.

A particularly preferred use of the method according to the invention is the use in diagnosing cancer diseases or other diseases associated with a change of the methylation status. Inter alia, these include CNS dysfunction, symptoms of aggression or behavioral disorders; clinical, psychological and social consequences of cerebral lesions, psychotic disorders and personality disorders; dementia and/or associated syndromes; cardiovascular diseases, dysfunctions and lesions; dysfunctions, lesions or diseases of the gastrointestinal tract; dysfunctions, lesions or diseases of the respiratory system; lesion, inflammation, infection, immunity and/or convalescence; dysfunctions, lesions or diseases of the body as a deviation in the process of development; dysfunctions, lesions or diseases of the skin, muscles, connective tissue or bones; endocrine and metabolic dysfunctions, lesions or diseases; headaches or sexual dysfunctions.

In addition, the method according to the invention is suitable for predicting undesirable drug effects and differentiating cell types or tissues or investigating cell differentiation.

Finally, the invention also relates to a kit which consists of at least one reagent for enrichment of the sequence to be analyzed and reagents for bisulfite conversion, and optionally also includes a polymerase, primers and probes for amplification and detection.

As reagents used to enrich the sequence to be analyzed, proteins binding in a methylation-specific manner are possible, in particular, which are preferably column-bound, biotinylated or provided with a histidine tag. Furthermore, modified PNA or DNA oligomers can be used, which are preferably immobilized, biotinylated or modified with magnetic particles.

1. (canceled)

2. (canceled)

3. The method according to claim 25, wherein the biological sample includes tissue samples or body fluids.

4. The method according to claim 25, wherein said enriching proceeds in a methylation-specific fashion.

5. The method according to claim 4, wherein said enriching is effected using proteins binding to the DNA in a methylation-specific fashion.

6. The method according to claim 5, wherein said enriching is effected using MeCP2, MBD1, MBD2, MBD4 or Kaiso.

7. The method according to claim 6, wherein said enriching is effected using MBD column chromatography.

8. The method according to claim 5, wherein said enriching is effected using methylation-specific antibodies.

9. The method according to claim 25, wherein said enriching is effected in a sequence-specific manner.

10. The method according to claim 25, wherein said enriching is effected according to the origin of the DNA.

11. The method according to claim 10, wherein the DNA is enriched from cancer cells in body fluids.

12. The method according to claim 11, wherein the DNA is bound to subcellular fragments.

13. The method according to claim 12, wherein the DNA is bound to nucleosomes.

14. The method according to claim 25, wherein said conversion is effected in the form of a bisulfite conversion.

15. The method according to claim 25, wherein said amplifying is effected using one of the following procedures: Heavy Methyl™, MSP; MethyLight™ or QM.

16. The method according to claim 25, wherein said enriching comprises at least two separate enrichment steps of the DNA.

17. (canceled)

18. (canceled)

19. A method for sensitive detection of cytosine methylation, comprising the following steps:
(a) collecting a biological sample,
(b) enriching the DNA to be detected, using sequence-specific capturing,
(c) detecting the enriched DNA via a methylation-specific antibody.

20. Use of the method according to claim 25 in the diagnosis of cancer diseases or other diseases associated with a change of the methylation status.

21. Use of the method according to claim 25 to predict undesirable drug effects, differentiate cell types and tissues or investigate cell differentiation.

22. A kit comprising at least one reagent for enrichment of the sequence to be analyzed and reagents for bisulfite conversion, and optionally also comprising a polymerase, primers and probes for amplification and detection.

23. The kit according to claim 22, wherein the reagents used in enrichment are proteins binding in a methylation-specific manner, which are column-bound, biotinylated or provided with a histidine tag.

24. The kit according to claim 22, wherein the reagents used in enrichment are modified PNA or DNA oligomers which are immobilized, biotinylated or modified with magnetic particles.

25. A method for sensitive methylation analysis, comprising the following steps:
(a) collecting a biological sample,
(b) (i) enriching the DNA to be detected of the biological sample, and
(ii) performing methylation-specific conversion of the DNA of the biological sample by chemical or enzymatic treatment, wherein steps (i) and (ii) can be performed in any order,
(c) amplifying the DNA from step (b),
(d) analyzing the amplified products.

26. The method according to claim 25 wherein step (b) (ii) follows step (b) (i).

27. The method according to claim 25 wherein step (b) (i) follows step (b) (ii).
